(19) United States
(12) Patent Application Publication
(10) Pub. No.: US 2025/0127463 A1
(43) Pub. Date: Apr. 24, 2025

(54) MOTION STATE MONITORING SYSTEM, CONTROL METHOD, AND NON-TRANSITORY COMPUTER READABLE MEDIUM

(71) Applicant: TOYOTA JIDOSHA KABUSHIKI KAISHA, Toyota-shi (JP)

(72) Inventors: Makoto Kobayashi, Nisshin-shi (JP); Hiroto Ichikawa, Seto-shi (JP); Toshiaki Okumura, Toyota-shi (JP)

(73) Assignee: TOYOTA JIDOSHA KABUSHIKI KAISHA, Toyota-shi (JP)

(21) Appl. No.: 18/830,777

(22) Filed: Sep. 11, 2024

(30) Foreign Application Priority Data
Oct. 24, 2023 (JP) 2023-182856

(51) Int. Cl. A61B 5/00; A61B 5/11

(52) U.S. Cl. CPC A61B 5/7435; A61B 5/1116; A61B 5/1121; A61B 5/6841; A61B 2560/0223

(57) ABSTRACT

A motion state monitoring system includes: a plurality of sensors attached to respective body parts of a plurality of respective subjects; and a plurality of motion state monitoring apparatuses. Each of the plurality of motion state monitoring apparatuses comprises: an icon display unit configured to display a plurality of sensor icons corresponding to the plurality of respective sensors; a display control unit configured to cause the icon display unit to display a sensor a distance of which from the motion state monitoring apparatus is within a predetermined distance among the plurality of sensors; a reception unit configured to receive a setting operation on a sensor icon displayed on the icon display unit; and a process unit configured to make, in accordance with the setting operation, the sensor corresponding to the sensor icon correspond to the body part of the subject to which the sensor is to be attached.

### CROSS REFERENCE TO RELATED APPLICATIONS

This application is based upon and claims the benefit of priority from Japanese patent application No. 2023-182856, filed on Oct. 24, 2023, the disclosure of which is incorporated herein in its entirety by reference.

### BACKGROUND

The present disclosure relates to a motion state monitoring system, a control method, and a program.

Japanese Unexamined Patent Application Publication No. 2022-034449 discloses a motion state monitoring system including a plurality of sensors corresponding to a plurality of respective body parts of a body of a subject, and a motion state monitoring apparatus. A plurality of sensors are made to correspond to the respective body parts by pairing processing performed between the sensors and the motion state monitoring apparatus. In this motion state monitoring system, a sensor is selected based on a motion to be monitored and a motion state of a subject is monitored based on a result of detection performed by the selected sensor.

### SUMMARY

When motions of a plurality of subjects are monitored, a set including a plurality of sensors where body parts to which these sensors are to be attached are set is prepared for each subject, and these sets are used by different respective motion state monitoring apparatuses. In this manner, the body parts to which the plurality of sensors in each set are to be attached are set for each subject. Therefore, even if a sensor in one set fails, a sensor of another set cannot be used in place of the failed sensor. Further, in order to use a sensor of another set, an operation for changing the body part to which the sensor is attached corresponding to the sensor needs to be performed, which causes a problem that it takes a lot of time and effort to perform the same.

It has therefore been desired to share sensors among a plurality of motion state monitoring apparatuses in a simple manner. It is possible that inference of motion states of the subjects using the above motion state monitoring system may be inference, for example, inference thereof using a learning model generated by machine learning or the like.

The present disclosure has been made in view of the aforementioned circumstances and an object of the present disclosure is to provide a motion state monitoring system, a control method, and a program capable of sharing a plurality of sensors in a plurality of motion state monitoring apparatuses in a simple manner.

A motion state monitoring system according to one aspect is a motion state monitoring system including a plurality of sensors attached to respective body parts of bodies of a plurality of respective subjects; and a plurality of motion state monitoring apparatuses configured to monitor the respective subjects by the attached sensors, in which each of the plurality of motion state monitoring apparatuses includes: an icon display unit configured to display a plurality of sensor icons corresponding to the plurality of respective sensors; a display control unit configured to cause the icon display unit to display a sensor a distance of which from the motion state monitoring apparatus is within a predetermined distance among the plurality of sensors; a reception unit configured to receive a setting operation on a sensor icon displayed on the icon display unit; and a process unit configured to make, in accordance with the setting operation, the sensor corresponding to the sensor icon correspond to the body part of the subject to which the sensor is to be attached.

A control method according to one aspect is a method for controlling a motion state monitoring system including a plurality of sensors attached to respective body parts of bodies of a plurality of respective subjects; and a plurality of motion state monitoring apparatuses configured to monitor the respective subjects by the attached sensors, in which each of the plurality of motion state monitoring apparatuses executes: processing for causing an icon display unit to display a plurality of sensor icons corresponding to respective sensors distances of which from the motion state monitoring apparatus are each within a predetermined distance among the plurality of sensors; processing for receiving a setting operation on the sensor icons displayed on the icon display unit; and processing for making, in accordance with the setting operation, the sensor corresponding to the sensor icon correspond to the body part of the subject to which the sensor is to be attached.

A program according to one aspect is a program for controlling a motion state monitoring system including a plurality of sensors attached to respective body parts of bodies of a plurality of respective subjects; and a plurality of motion state monitoring apparatuses configured to monitor the respective subjects by the attached sensors, and the program causes each of the plurality of motion state monitoring apparatuses to execute: processing for causing an icon display unit to display a plurality of sensor icons corresponding to respective sensors distances of which from the motion state monitoring apparatus are each within a predetermined distance among the plurality of sensors; processing for receiving a setting operation on the sensor icons displayed on the icon display unit; and processing for making, in accordance with the setting operation, the sensor corresponding to the sensor icon correspond to the body part of the subject to which the sensor is to be attached.

According to the present disclosure, it becomes possible to share a plurality of sensors in a plurality of motion state monitoring apparatuses in a simple manner.

The above and other objects, features and advantages of the present disclosure will become more fully understood from the detailed description given hereinbelow and the accompanying drawings.

### BRIEF DESCRIPTION OF DRAWINGS

FIG. 1 is a block diagram showing a configuration example of a training support system according to an embodiment;

FIG. 2 is a diagram showing one example of body parts to which measuring instruments are to be attached;

FIG. 3 is a diagram showing a configuration example of a measuring instrument provided in the training support system shown in FIG. 1;

FIG. 4 is a diagram showing an example of how to attach the measuring instrument shown in FIG. 3;

FIG. 5 is a diagram showing one example of a display screen;

FIG. 6 is a flowchart showing a motion of one motion state monitoring apparatus in the training support system shown in FIG. 1; and

FIG. 7 is a flowchart showing a motion of another motion state monitoring apparatus in the training support system shown in FIG. 1.

### DESCRIPTION OF EMBODIMENTS

Hereinafter, embodiments of the present disclosure will be described with reference to the accompanying drawings. For a purpose of clarifying the description, the following description and the drawings will be omitted and simplified as appropriate. Throughout the drawings, the same symbols are attached to the same elements and overlapping descriptions are omitted as necessary. Hereinafter, in embodiments described below, when a number like a number of a certain thing, quantity, amount, range, or the like of each element is referred to, the idea relating to the present disclosure is not limited to the number referred to, except when it is particularly clarified, or the number is clearly specified in principle. In addition, the structure and the like in the embodiments described below are not necessarily essential to the idea relating to the present disclosure, except when particularly clarified or explicitly specified in principle.

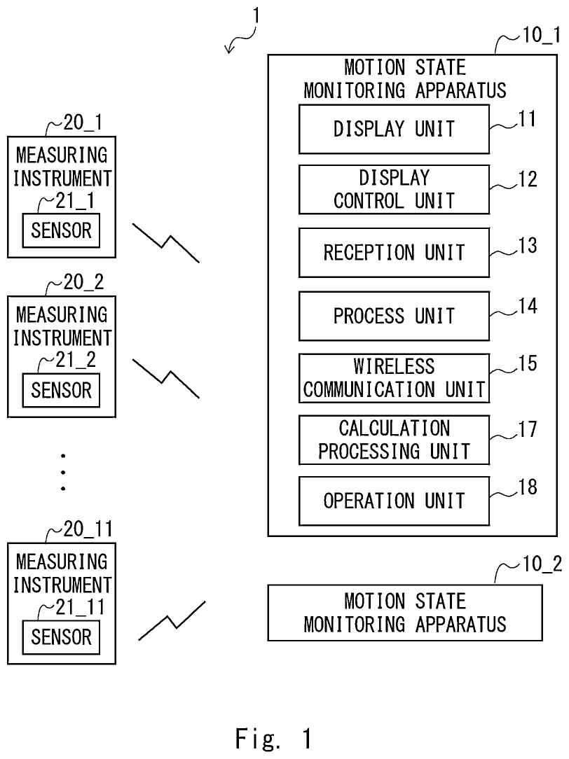

FIG. 1 is a block diagram showing a configuration example of a training support system 1 according to an embodiment. The training support system 1 is a system for monitoring a motion of a subject and providing support for making the motion of the subject close to a desired motion based on a result of the monitoring. The training support system 1 is used, for example, by a user such as an assistant.

As shown in FIG. 1, the training support system 1 includes a plurality of motion state monitoring apparatuses 10 and a plurality of measuring instruments 20. In this embodiment, an example in which 11 measuring instruments 20 are shared by two motion state monitoring apparatuses 10 will be described. The training support system 1 can monitor a plurality of subjects P. The plurality of motion state monitoring apparatuses 10 monitor the plurality of respective subjects P. In the training support system 1 according to this embodiment, two subjects P may be monitored by two motion state monitoring apparatuses 10.

In the following description, the two motion state monitoring apparatuses 10 are also referred to as motion state monitoring apparatuses 10_1 and 10_2, respectively, in order to distinguish them from each other. It is assumed that the motion state monitoring apparatus 10_1 monitors a motion state of a subject P1 and the motion state monitoring apparatus 10_2 monitors a motion state of a subject P2. Further, the 11 measuring instruments 20 are also referred to as measuring instruments 20_1 to 20_11, respectively, in order to distinguish them from each other.

At least one of the 11 shared measuring instruments is used in one motion state monitoring apparatus 10_1 and at least one of the remaining measuring instruments is used in the other motion state monitoring apparatus 10_2. The measuring instruments 20_1 to 20_11 are attached to respective body parts p1 to p11 from which motions are to be detected among various body parts of bodies of a plurality of subjects P. Hereinafter, the body parts p1 to p11 to which the measuring instruments are attached will also be collectively referred to as a body part(s) p.

The measuring instruments 20_1 to 20_11 are made to correspond to the respective body parts p1 to p11 of the two subjects P1 and P2 as a result of correspondence processing performed with one of the motion state monitoring apparatuses 10_1 and 10_2. The measuring instruments 20_1 to 20_11 detect motions of the respective body parts p1 to p11 attached using motion sensors (hereinafter simply referred to as sensors) 21_1 to 21_11 such as gyro sensors. The motion state monitoring apparatuses 10_1 and 10_2 respectively monitor the motion states of the subjects P1 and P2 based on results of detection from the sensors of the measuring instruments 20 attached to the subjects P1 and P2. Hereinafter, the sensors 21_1 to 21_11 will be collectively referred to as a sensor(s) 21.

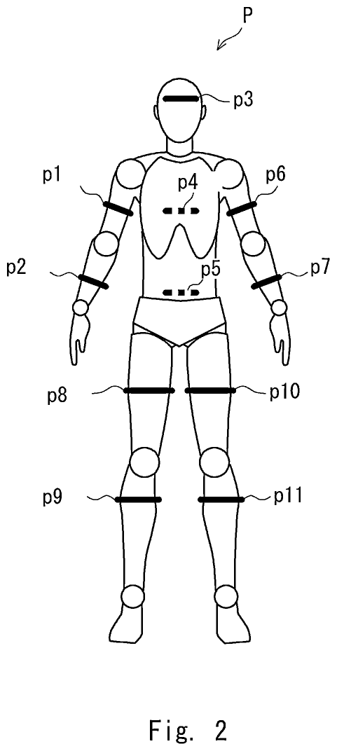

FIG. 2 is a diagram showing one example of the body parts p to which the measuring instruments 20 are to be attached. In the example shown in FIG. 2, the body parts p1 to p11 to which the measuring instruments 20 are to be attached are a right upper arm, a right forearm, a head, a back (a trunk), a waist (a pelvis), a left upper arm, a left forearm, a right thigh, a right lower leg, a left thigh, and a left lower leg, respectively. It is assumed, in this example, that the back and the waist are positioned on the back side of the subject P. The measuring instruments 20 are attached to at least one body part of the plurality of subjects P to which the measuring instruments 20 are to be attached.

The motions to be monitored include, for example, motions such as bending and stretching of the right shoulder, adduction and abduction of the right shoulder, internal and external rotation of the right shoulder, bending and stretching of the right elbow, pronation and supination of the right forearm, bending and stretching of the head, rotation of the head, bending and stretching of the chest and the waist, rotation of the chest and the waist, lateral bending of the chest and the waist, bending and stretching of the left shoulder, adduction and abduction of the left shoulder, internal and external rotation of the left shoulder, bending and stretching of the left elbow, pronation and supination of the left forearm. Further, the motions to be monitored include the motion of the body part itself to which a sensor is attached.

For example, the motions to be monitored include angles of joints of the body of the subject P measured based on results of detection performed by the plurality of sensors or angles of joints in any coordinate system measured based on a result of detection performed by any one of the sensors. The motions to be monitored may be specified by a user such as an assistant.

It is assumed here, as one example, that the bending and stretching motion of the right elbow of each of the two subjects P1 and P2 is to be monitored. The bending and stretching motion of the right elbow can be measured based on a result of detection performed by each of the sensors attached to the right upper arm (the body part p1) and the right forearm (the body part p2). In this case, for example, two measuring instruments are respectively attached to the right upper arm (the body part p1) and the right forearm (the body part p2) of one subject P1, and two different measuring instruments are respectively attached to the right upper arm (the body part p1) and the right forearm (the body part p2) of the other subject P2.

Note that the user may concurrently select items of a plurality of different motions to be monitored. For example, the user may select “bending and stretching of the right elbow” and “internal and external rotation of the right shoulder” in the motion state monitoring apparatus 10_1 and select “bending and stretching of the left elbow” and “internal and external rotation of the left shoulder” in the motion state monitoring apparatus 10_2.

The bending and stretching motion of the right elbow may be measured based on results of detection performed by the respective sensors attached to the right upper arm (the body part p1) and the right forearm (the body part p2). Likewise, the internal and external rotation motion of the right shoulder may be measured based on results of detection performed by the respective sensors attached to the right upper arm (the body part p1) and the right forearm (the body part p2). Further, the bending and stretching motion of the left elbow may be measured based on results of detection performed by the respective sensors attached to the left upper arm (the body part p6) and the left forearm (the body part p7). Likewise, the internal and external rotation motion of the left shoulder may be measured based on results of detection performed by the respective sensors attached to the left upper arm (the body part p6) and the left forearm (the body part p7).

### (Configuration Example of Measuring Instruments 20_1 to 20_11)

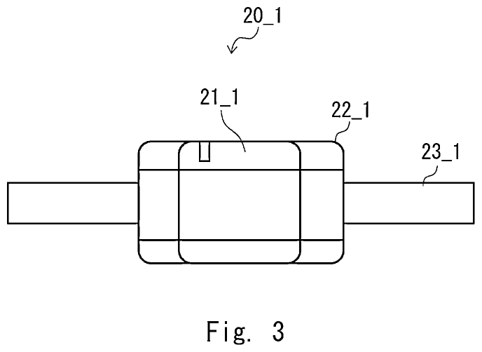

FIG. 3 is a diagram showing a configuration example of the measuring instrument 20_1. Note that the configuration of each of the measuring instruments 20_2 to 20_11 is similar to that of the measuring instrument 20_1, and thus the descriptions thereof will be omitted.

As shown in FIG. 3, the measuring instrument 20_1 includes a sensor 21_1, an attachment pad 22_1, and a belt 23_1. The belt 23_1 is configured so that it can be wound around the body part of the subject P from which a motion is to be detected. The sensor 21_1 is integrated with, for example, the attachment pad 22_1. Further, the attachment pad 22_1 is configured so that it can be attached to or detached from the belt 23_1.

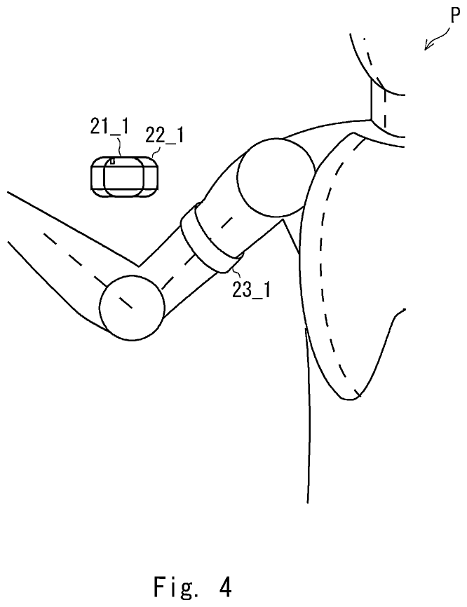

FIG. 4 is a diagram showing an example of how to attach the measuring instrument 20_1. In the example shown in FIG. 4, the belt 23_1 is wound around the right upper arm (the body part p1), which is one of the body parts of the subject P from which motions are to be detected. The sensor 21_1 is attached to the belt 23_1 with the attachment pad 22_1 interposed therebetween after correspondence processing, calibration, and the like have been completed.

### (Configuration Example of Motion State Monitoring Apparatus 10)

The motion state monitoring apparatus 10 is an apparatus that outputs a result of a calculation indicating a motion state of the subject P based on results of detection (sensing values) performed by the sensors 21_1 to 21_11. The motion state monitoring apparatus 10 may be, for example, a Personal Computer (PC), a mobile phone terminal, a smartphone, a tablet terminal, or the like. The motion state monitoring apparatus 10 is configured to be able to communicate with the sensors 21_1 to 21_11 via a network (not shown). The motion state monitoring apparatus 10 may also be referred to as a motion state monitoring system.

Since the motion state monitoring apparatus 10_2 has a configuration the same as that of the motion state monitoring apparatus 10_1, the detailed configuration of the motion state monitoring apparatus 10_2 is not shown in FIG. 1. Hereinafter, a configuration of the motion state monitoring apparatus 10_1 will be described.

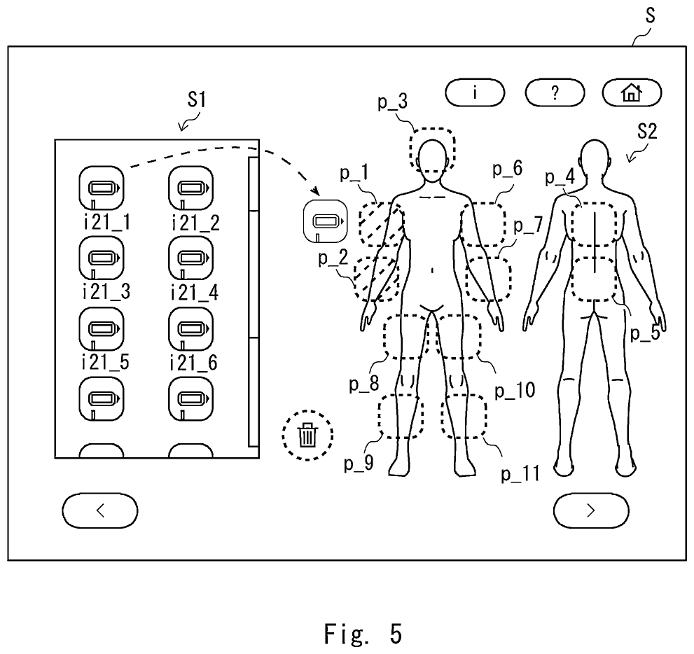

As shown in FIG. 1, the motion state monitoring apparatus 10_1 includes a display unit 11, a display control unit 12, a reception unit 13, a process unit 14, a wireless communication unit 15, a calculation processing unit 17, and an operation unit 18. The display unit 11 is, for example, a display apparatus, and displays a plurality of sensor icons corresponding to the plurality of respective sensors 21_1 to 21_11. FIG. 5 shows one example of a display screen S displayed on the display unit 11. The area of the display screen S where sensor icons are displayed is denoted by a sensor icon display area S1. Further, the display unit 11 displays, when it receives an operation performed by a user, an input screen of information on the subject, a screen for selecting a result of the monitoring to be displayed on the display unit 11, or a result of the monitoring generated after the motion state of the subject is monitored.

Note that the display screen including the sensor icon display area S includes a human body schematic diagram S2 showing body parts to which sensors are to be attached, as will be described later. This human body schematic diagram S2 is an attachment target body part display unit in which body parts to which sensors are to be attached are shown on a diagram showing a human body. The display screen S shown in FIG. 5 is displayed when processing for making one of the plurality of sensors 21 correspond to one of the body parts p of the subject P to which the sensors are to be attached in one-to-one is performed.

In the example shown in FIG. 5, the front side and the back side of the human body are displayed in a separated way. The body parts p1 to p11 of the subject P to which sensors are to be attached in FIG. 2 respectively correspond to p_1 to p_11 of the human body schematic diagram S2 in FIG. 5. Hereinafter, as necessary, p_1 to p_11 of the human body schematic diagram S2 are respectively referred to as a right upper arm, a right forearm, a head, a back (a trunk), a waist (a pelvis), a left upper arm, a left forearm, a right thigh, a right lower leg, a left thigh, and a left lower leg. Further, the display unit 11 is able to display the result of the calculation based on the result of the detection performed by each of one or more sensors in a form of a graph, for example.

The display control unit 12 causes the display unit 11 to display some of the plurality of sensors 21 distances of which from the motion state monitoring apparatus 10_1 are each within a predetermined distance. For example, the wireless communication unit 15 is able to receive identification information from each of the plurality of sensors 21 located within a predetermined distance by short-range wireless communication. The display control unit 12 causes the display unit 11 to display sensor icons different for each sensor based on the received identification information. That is, the display unit 11 displays a list of sensor icons corresponding to the respective available sensors that have not been made to correspond to any body part to which the sensor is to be attached.

It is assumed here that the wireless communication unit 15 performs data communication conforming to Bluetooth (registered trademark) standard as the short-range wireless communication. The motion state monitoring apparatus 10 and a sensor 21 that is present within a communication range are paired with each other by exchanging identification information such as a Bluetooth address and mutually authenticating with each other, whereby the motion state monitoring apparatus 10 is connected to the sensor 21. Once the pairing is completed, necessary information is stored in each other's equipment. In the following processing, when the sensor 21 is located within a predetermined distance from the motion state monitoring apparatus 10, they are connected to each other without performing pairing.

Note that the wireless communication unit 15 may instead perform short-range wireless communication conforming to Near field communication (NFC), Ultra Wideband (UWB), WiFi (registered trademark), or the like. Note that the aforementioned predetermined distance may vary depending on the type of the short-range wireless communication to be adopted.

Note that the motion state monitoring apparatus 10_1 may include a distance measurement sensor (not shown) such as Light Detection and Ranging, Laser Imaging Detection and Ranging (LiDAR) that uses a pulsed laser light as a measurement signal. The distance measurement sensor is able to measure the distance to a sensor that is present near the motion state monitoring apparatus 10. The display control unit 12 can cause the display unit 11 to display sensors distances of which from the motion state monitoring apparatus 10_1 are each within a predetermined distance based on the result of measurement in the distance measurement sensor.

The above plurality of sensor icons are selected by the user in order to make the sensor 21 correspond to the body part p to which the sensor is to be attached. The operation unit 18 may include an input apparatus such as a mouse or a keyboard. The operation unit 18 may be a touch panel in which a display apparatus and an input apparatus are integrated with each other. For example, the user is able to select a plurality of sensor icons by operating a mouse, a keyboard or the like of the operation unit 18 or operating a touch panel of the operation unit 18 with a touch pen or his/her finger.

The reception unit 13 receives a setting operation on a sensor icon displayed on the display unit 11. The setting operation means an input operation on the sensor icon for making the sensor corresponding to the sensor icon correspond to any body part p to which the sensor is to be attached. For example, the user moves a sensor icon i21_1 displayed in the sensor icon display area S1 to the right upper arm p_1 on the human body schematic diagram S2 by operating the operation unit 18. Specifically, the user drags and drops the sensor icon i21_1 displayed in the sensor icon display area S1 onto the right upper arm p_1 in the human body schematic diagram S2 by a mouse operation, a touch operation, or the like. This setting operation is shown by a dotted line in FIG. 5. Accordingly, the reception unit 13 receives the setting operation on the sensor icon performed by the user.

The plurality of sensors 21 may each include a light-emitting unit capable of changing a luminescence color thereof. The light-emitting unit may be, for example, a full-color LED. As one example, the light-emitting unit may have a configuration in which a red LED chip, a green LED chip, and a blue LED chip are sealed by resin having optical transparency. The light-emitting unit can provide a wide variety of luminescence colors by controlling brightness of three kinds of LED chips.

The motion state monitoring apparatus 10_1 may further include a color control unit configured to execute processing for making the display colors of the plurality of sensor icons the same as the luminescence colors of the light-emitting units of the plurality of respective sensors. The user can select a sensor of the luminescence color which is the same as the display color of the sensor icon, check the human body schematic diagram S2, and actually attach this sensor to the body part p of the subject P to which the sensor is to be attached. Accordingly, it becomes possible to prevent a wrong sensor from being attached.

The process unit 14 makes, in accordance with the setting operation, the sensor corresponding to the sensor icon correspond to the body part of the subject to which the sensor is to be attached. This correspondence processing is performed by pairing processing performed between the motion state monitoring apparatus 10 and the sensor 21 in advance, and making identification information of the body part p to which the sensor is to be attached correspond to identification information of the sensor 21 on the application of the motion state monitoring apparatus 10. Note that, after the body part p to which the sensor is to be attached is made to correspond to the sensor 21, the user can perform processing for canceling this correspondence.

As described above, according to the present disclosure, the user can set correspondence of sensors 21 which are each located within a predetermined distance from the plurality of motion state monitoring apparatuses 10 with body parts p to which the sensors 21 are to be attached in any one of the motion state monitoring apparatuses 10 in a free and simple operation. Accordingly, the sensors 21 can be shared among the plurality of motion state monitoring apparatuses 10, whereby a highly convenient system can be achieved.

The calculation processing unit 17 performs calculation processing based on a result of detection performed by each of the sensors 21_1 to 21_11 to generate a result of the calculation indicating the motion state of the motion to be monitored of the subject P. Note that the calculation processing unit 17 may perform calculation processing by using a learned model generated by machine learning performed using previous results of detection performed in sensors. The calculation processing unit 17 may accurately calculate whether or not the motion state of the motion to be monitored of the subject P is fine by performing calculation processing using the above learned model. The calculation processing unit 17 transmits the result of the calculation to the display control unit 12.

The display control unit 12 causes the display unit 11 to display the information received from the calculation processing unit 17 (the result of the calculation) in such a manner that this information can be visualized in a form of a graph or the like. Accordingly, the user is able to know the motion state of the motion to be monitored of the subject P, and use the obtained information to assist the subject P, for example.

Note that the setting operation is not limited to the drag and drop operation. The user clicks a sensor icon displayed in the sensor icon display area S1 and the body part to which the sensor is to be attached on the human body schematic diagram S2 within a predetermined period of time, whereby the reception unit 13 may receive the setting operation.

Further, the user clicks the body part to which the sensor is to be attached on the human body schematic diagram S2, whereby the reception unit 13 may receive the setting operation. The process unit 14 may able to automatically make, in accordance with the setting operation, one of the sensors corresponding to the plurality of sensor icons displayed on the display unit 11 correspond to the body part p of the subject P to which the sensor is to be attached. For example, the process unit 14 may perform paring of a plurality of sensors that do not correspond to any of the body parts p of the subject P to which sensors are to be attached in accordance with an ascending order of respective distances of the sensors from the motion state monitoring apparatus 10_1 to make each sensor correspond to any of the body parts p of the subject P to which this sensor is to be attached.

Further, a sensor icon corresponding to one sensor may be displayed in the plurality of motion state monitoring apparatuses 10_1 and 10_2. When the motion state monitoring apparatuses 10_1 and 10_2 have concurrently detected the same sensor, it is possible to grasp a situation in which the motion state monitoring apparatuses 10_1 and 10_2 concurrently detect the same sensor. It is assumed, for example, that the sensor icon i21_1 corresponding to the same sensor 21_1 is displayed. In this case, when the correspondence of the sensor 21_1 with the body part p1 of the subject P1 to which the sensor is to be attached is performed first in one motion state monitoring apparatus 10_1, the display control unit 12 in the other motion state monitoring apparatus 10_2 performs processing for deleting the sensor icon i21_1 corresponding to the sensor 21_1 that has already been made to correspond to the body part p1.

That is, the sensor icon i21_1 is not displayed on the display unit 11 of the motion state monitoring apparatus 10_2. In other words, only the sensor icons i21-2 to i21_11 corresponding to available sensors remain in the display unit 11 of the motion state monitoring apparatus 10_2. When the same sensor icon is displayed in two motion state monitoring apparatuses 10, the user may delete the sensor icon in one motion state monitoring apparatus 10 in order to prevent each motion state monitoring apparatus 10 from performing a setting operation on the same sensor. For example, the user can delete the sensor icon by dragging and dropping the sensor icon onto a trash box icon.

Alternatively, in the motion state monitoring apparatus 10_2, the display control unit 12 may disable the sensor icon i21_1 so as to prevent the user from performing a setting operation on this sensor icon i21_1. That is, in the display unit 11 of the motion state monitoring apparatus 10_2, only the sensor icons i21-2 to i21_11 corresponding to the available sensors are in a state in which they can receive the setting operation. Accordingly, it is possible to prevent one sensor from being set in a plurality of motion state monitoring apparatuses 10 in a redundant manner.

Further, in the motion state monitoring apparatus 10_1, it is possible that the sensor 21_1 corresponding to the right upper arm (the body part p1) of the subject P1 may fail while it is being used and may be in an inoperable state. In this case, the user is able to perform a re-setting operation on a sensor icon corresponding to the available sensor that has been displayed in the sensor icon display area S1 and has not been made to correspond to any sensor. Accordingly, the reception unit 13 is able to receive the re-setting operation on the sensor icon performed by the user.

Specifically, among the sensors 21_1 to 21_11, the sensors 21_5 to 21_11 other than the sensors 21_1 and 21_2 in which the correspondence processing is performed in the motion state monitoring apparatus 10_1, and the sensors 21_3 and 21_4 in which the correspondence processing is performed in the motion state monitoring apparatus 10_2 can be used.

For example, the user drags and drops the sensor icon i21_5, which is one of the sensor icons i21_5 to i21_11 corresponding to the sensors 21_5 to 21_11, onto the right upper arm p_1 in the human body schematic diagram S2. Accordingly, the reception unit 13 receives a re-setting operation on the sensor icon i21_5 performed by the user. The process unit 14 is able to make the sensor 21_5 corresponding to the sensor icon i21_5 correspond to the right upper arm (the body part p1) of the subject P1 instead of making the sensor 21_1 which is in an inoperable state correspond to the right upper arm of the subject P1.

Accordingly, it becomes possible to change the correspondence of a sensor with a body part to which the sensor is to be attached by a simple operation. Before the processing for making the other sensor 21_5 correspond to the right upper arm p1 of the subject is performed, the user may perform an input operation for releasing the correspondence of the sensor 21_1 with the right upper arm (the body part p1) of the subject.

### (Motions of Training Support System 1)

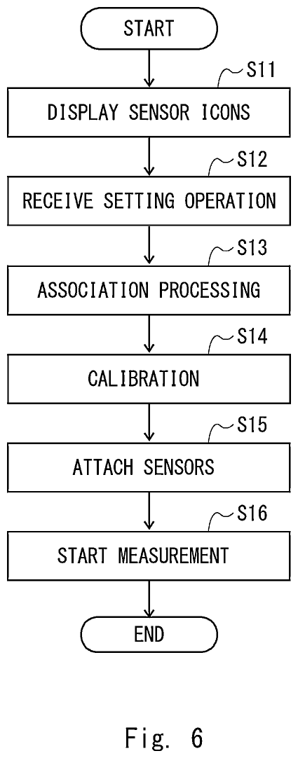

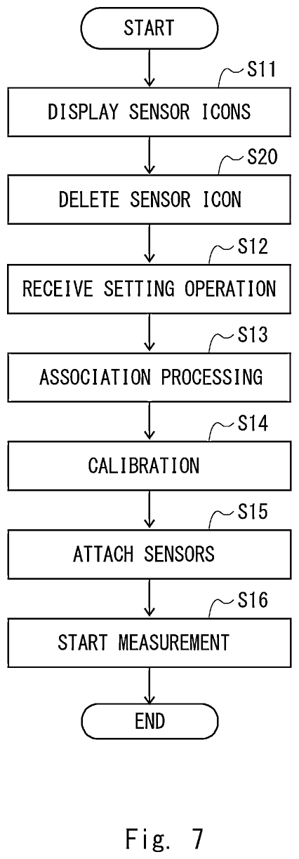

FIGS. 6 and 7 are flowcharts showing motions of the training support system 1. As described above, it is assumed here that the bending and stretching motion of the right elbow of the subject P1 and that of the subject P2 are respectively monitored by the motion state monitoring apparatuses 10_1 and 10_2. That is, sensors are attached to the right upper arm (the body part p1) and the right forearm (the body part p2) of the subject P1 and sensors are attached to the right upper arm (the body part p1) and the right forearm (the body part p2) of the subject P2. Accordingly, two sensors are used in the motion state monitoring apparatus 10_1 and two sensors that are different from the above sensors are used in the motion state monitoring apparatus 10_2.

In the following example, it is assumed that processing for making the sensors correspond to the body parts to which the respective sensors are to be attached is performed first in the motion state monitoring apparatus 10_1 and then processing for making the sensors correspond to the body parts to which the respective sensors are to be attached is performed in the motion state monitoring apparatus 10_2. FIG. 6 shows the motions of the motion state monitoring apparatus 10_1. Further, FIG. 7 shows the motions of the motion state monitoring apparatus 10_2. In FIGS. 6 and 7, the same processes are denoted by the same reference symbols.

First, the motion state monitoring apparatuses 10_1 and 10_2 cause the display unit 11 to display sensor icons i21_1 to i21_11 corresponding to the plurality of respective sensors that are each located within a predetermined distance (S11). At this time, not all the sensor icons corresponding to all the sensors are displayed in the motion state monitoring apparatuses 10_1 and 10_2 and some sensor icons may not be displayed depending on the distances from the motion state monitoring apparatuses 10_1 and 10_2.

It is assumed here, as one example, that all the sensors 21_1 to 21_11 are each located within a predetermined distance from both the motion state monitoring apparatuses 10_1 and 10_2. It is therefore assumed that all the sensor icons i21_1 to i21_1 respectively corresponding to all the sensors 21_1 to 21_11 are displayed in both the motion state monitoring apparatuses 10_1 and 10_2.

The display unit 11 displays a display screen including the sensor icon display area S1 which displays sensor icons corresponding to the sensors distances of which from the motion state monitoring apparatus 10 are each within a predetermined distance and the human body schematic diagram S2 in which the body parts to which the sensors are to be attached are displayed, as shown in FIG. 5.

When the user specifies the motions to be monitored of the subject P, the display unit 11 may display the body parts to which sensors used to measure the specified motions to be monitored are to be attached. The display unit 11 of each of the motion state monitoring apparatuses 10_1 and 10_2 may highlight the right upper arm p_1 and the right forearm p_2 in the human body schematic diagram S2 to indicate that the right upper arm p_1 and the right forearm p2 have a display aspect (color, flashing, shading, or the like) different from those of the other body parts p_3 to p_11. That is, in the human body schematic diagram S2 shown in FIG. 5, the right upper arm p_1 and the right forearm p_2 may have a display aspect different from those of the other body parts p_3 to_p11.

Then, in the motion state monitoring apparatus 10_1, the user drags and drops, for example, one sensor icon i21_1 onto the right upper arm p_1 in the human body schematic diagram S2 from the sensor icons 121_1 to i21_11 displayed in the sensor icon display area S1. Accordingly, the reception unit 13 receives the setting operation (S12).

After that, the motion state monitoring apparatus 10_1 makes, in accordance with the setting operation, identification information of the sensor 21_1 corresponding to the sensor icon i21_1 correspond to identification information of the right upper arm (the body part p1) of the subject P1 to which the sensor is to be attached. Accordingly, the processing for making the sensor 21_1 correspond to the right upper arm (the body part p1) of the subject P1 is performed (S13). Drag and drop of the sensor icon i21_2 and processing for making the sensor 21_2 correspond to the right forearm (the body part p2) of the subject P1 are performed for another body part to which a sensor is to be attached (right forearm (the body part p2)) as well. That is, S12 and S13 in FIG. 6 may be repeated for the number of body parts of the subject P1 to which sensors are to be attached.

After the correspondence of the sensor 21_1 with the body part p1 to which the sensor 21_1 is to be attached and the correspondence of the sensor 21_2 with the body part p2 to which the sensor 21_2 is to be attached are completed, calibration of the sensors 21_1 and 21_2 used to measure the motions to be monitored is performed (S14). The calibration indicates, for example, processing for measuring an output value (error component) in a stationary state of the sensor used to measure the motion to be monitored and subtracting an error component from the measured value. In this example, calibration of at least the sensors 21_1 and 21_2 is performed. However, the calibration may be performed on not only the sensor used to measure the motion to be monitored but also all the sensors 21_1 to 21_11 at a timing before, for example, the processing for displaying the sensor icon corresponding to the sensor that has already been subjected to pairing.

After the calibration is completed, the sensors 21_1 and 21_2 are attached to the subject P1 (S15). After that, the motion to be monitored is measured based on a result of detection performed by each of the sensors 21_1 and 21_2 (S16).

The result of the calculation indicating the motion state of “bending and stretching of the right elbow” can be calculated from the difference between the result of the detection performed in the sensor 21_1 attached to the right upper arm (the body part p1) of the subject P1 and the result of the detection performed in the sensor 21_2 attached to the right forearm (the body part p2). The motion state monitoring apparatus 10_1 generates the result of the calculation indicating the motion state of “bending and stretching of the right elbow” based on the result of the detection performed by each of the sensors 21_1 and 21_2. The display unit 11 displays the details of the result of the measurement (e.g., a result of measurement shown in a form of a graph).

After the processing for making the sensors 21_1 and 21_2 correspond to the body parts to which the sensors are to be attached in the motion state monitoring apparatus 10_1 is completed, the motion state monitoring apparatus 10_2 performs processing for deleting the sensor icons i21_1 and i21_2 corresponding to the sensors 21_1 and 21_2 set in the motion state monitoring apparatus 10_1 among the sensor icons i21_1 to i21_11 displayed on the display unit 11 (S20). Accordingly, in the sensor icon display area S1 of the display unit 11 of the motion state monitoring apparatus 10_2, available sensor icons i21_3 to i21_11 other than the sensor icons i21_1 and i21_2 remain. Note that, as described above, processing for disabling the sensor icons i21_1 and i21_2 may be performed.

Then, in the motion state monitoring apparatus 10_2, the user drags and drops, for example, one sensor icon i21_3 from the sensor icons i21_3 to i21_11 displayed in the sensor icon display area S1 onto the right upper arm p_1 in the human body schematic diagram S2. Accordingly, the reception unit 13 receives the setting operation (S12).

After that, the motion state monitoring apparatus 10_2 makes, in accordance with the setting operation, identification information of the sensor 21_3 corresponding to the sensor icon i21_3 correspond to identification information on the right upper arm (the body part p1) of the subject P2 to which this sensor is attached. Accordingly, processing for making the sensor 21_3 correspond to the right upper arm (the body part p1) of the subject P2 is performed (S13). The drag and drop of the sensor icon i21_4 and the processing for making the sensor 21_4 correspond to the right forearm (the body part p2) of the subject P2 are performed for another body part to which a sensor is to be attached (right forearm (the body part p2)) as well. That is, S12 and S13 in FIG. 7 may be repeated for the number of body parts of the subject P2 to which sensors are to be attached. Hereinafter, as described in FIG. 6, processing from S14 to S16 is executed.

As described above, the training support system 1 according to this embodiment includes a plurality of motion state monitoring apparatuses 10 that display a plurality of sensor icons corresponding to a plurality of sensors located within a predetermined distance. Then, each of the motion state monitoring apparatuses 10 makes, in accordance with the setting operation on the displayed sensor icon, the sensor corresponding to the sensor icon correspond to the body part of the subject to which the sensor is to be attached. Accordingly, the training support system 1 is able to share the plurality of sensors 21_1 to 21_11 among the plurality of motion state monitoring apparatuses 10, whereby a highly convenient system can be achieved.

Further, after the correspondence between the sensor with the body part of the subject to which the sensor is to be attached is completed in one motion state monitoring apparatus, the sensor icon corresponding to the sensor that has already been made to correspond to the body part is deleted in the other motion state monitoring apparatus, or the sensor icon is disabled so that the setting operation can no longer be performed in the other motion state monitoring apparatus. Accordingly, it is possible to prevent one sensor from being set in a plurality of motion state monitoring apparatuses 10 in a redundant manner.

Note that the order of the processing in the training support system 1 is not limited to the order of the processing shown in FIGS. 6 and 7. For example, the calibration may be performed prior to the processing for displaying the sensor icon corresponding to the sensor that has already been subjected to pairing.

While the present disclosure has been described as a hardware configuration in the aforementioned example embodiments, the present disclosure is not limited thereto. The present disclosure can achieve the processing for controlling the motion state monitoring apparatus by causing a Central Processing Unit (CPU) to execute a computer program.

Further, the above-described program can be stored and provided to a computer using any type of non-transitory computer readable media. Non-transitory computer readable media include any type of tangible storage media. Examples of non-transitory computer readable media include magnetic storage media, optical magnetic storage media, CD-Read Only Memory (ROM), CD-R, CD-R/W, semiconductor memories. Magnetic storage media include, for example, flexible disks, magnetic tapes, hard disk drives, etc. Optical magnetic storage media include, for example, magneto-optical disks. Semiconductor memories include, for example, mask ROM, Programmable ROM (PROM), Erasable PROM (EPROM), flash ROM, Random Access Memory (RAM), etc. The program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A motion state monitoring system comprising:
a plurality of sensors attached to respective body parts of bodies of a plurality of respective subjects; and
a plurality of motion state monitoring apparatuses configured to monitor the respective subjects by the attached sensors, wherein
each of the plurality of motion state monitoring apparatuses comprises:
an icon display unit configured to display a plurality of sensor icons corresponding to the plurality of respective sensors;
a display control unit configured to cause the icon display unit to display a sensor a distance of which from the motion state monitoring apparatus is within a predetermined distance among the plurality of sensors;
a reception unit configured to receive a setting operation on a sensor icon displayed on the icon display unit; and
a process unit configured to make, in accordance with the setting operation, the sensor corresponding to the sensor icon correspond to the body part of the subject to which the sensor is to be attached.

2. The motion state monitoring system according to claim 1, wherein
in a case where a sensor icon corresponding to one sensor is displayed in each of a plurality of motion state monitoring apparatuses,
when a sensor is made to correspond to a body part of a subject to which a sensor is to be attached in one motion state monitoring apparatus, a display control unit of another motion state monitoring apparatus deletes a sensor icon corresponding to the sensor that has already been made to correspond to the body part of the subject or disables the sensor icon in such a way that the setting operation can no longer been performed.

3. The motion state monitoring system according to claim 1, wherein
in a case where a state of a sensor that is made to correspond to the body part of the subject to which the sensor is to be attached becomes inoperable,
the reception unit receives a re-setting operation of a sensor icon corresponding to a sensor other than the sensor that is made to correspond to the body part of the subject to which the sensor is to be attached displayed on the icon display unit, and
the process unit makes, in place of the sensor that is in the inoperable state, a sensor corresponding to the sensor icon in accordance with the re-setting operation correspond to the body part of the subject to which the sensor is to be attached.

4. The motion state monitoring system according to claim 1, further comprising an attachment target body part display unit showing the body parts to which the sensors are to be attached on a diagram showing a human body,
wherein the reception unit receives the setting operation by dragging and dropping the sensor icon onto the body part to which the sensor is to be attached.

5. The motion state monitoring system according to claim 1, further comprising an attachment target body part display unit showing the body parts to which the sensors are to be attached on a diagram showing a human body,
wherein the reception unit receives the setting operation by clicking each of the sensor icon and the body part to which the sensor is to be attached.

6. The motion state monitoring system according to claim 1, further comprising an attachment target body part display unit showing body parts to which the sensors are to be attached on a diagram showing a human body, wherein
the reception unit receives the setting operation by clicking the body part to which the sensor is to be attached, and
the process unit automatically makes, in accordance with the setting operation, any one of the plurality of sensors displayed on the icon display unit correspond to the body part of the subject to which the sensor is to be attached.

7. The motion state monitoring system according to claim 6, wherein the process unit makes a plurality of sensors that do not correspond to the body parts of the subject correspond to the body parts of the subject to which the sensors are to be attached in accordance with an ascending order of respective distances of the sensors from the motion state monitoring apparatus.

8. The motion state monitoring system according to claim 1, wherein
each of the plurality of sensors comprises a light-emitting unit that is capable of changing a luminescence color, and
the motion state monitoring system further comprises a color control unit that executes processing for making display colors of the plurality of sensor icons the same as luminescence colors of the light-emitting units of the plurality of respective sensors.

9. A method for controlling a motion state monitoring system comprising:
a plurality of sensors attached to respective body parts of bodies of a plurality of respective subjects; and
a plurality of motion state monitoring apparatuses configured to monitor the respective subjects by the attached sensors, wherein
each of the plurality of motion state monitoring apparatuses executes:
processing for causing an icon display unit to display a plurality of sensor icons corresponding to respective sensors distances of which from the motion state monitoring apparatus are each within a predetermined distance among the plurality of sensors;
processing for receiving a setting operation on the sensor icons displayed on the icon display unit; and
processing for making, in accordance with the setting operation, the sensor corresponding to the sensor icon correspond to the body part of the subject to which the sensor is to be attached.

10. A non-transitory computer readable medium storing a program for controlling a motion state monitoring system comprising a plurality of sensors attached to respective body parts of bodies of a plurality of respective subjects and a plurality of motion state monitoring apparatuses configured to monitor the respective subjects by the attached sensors, wherein
the program causes each of the motion state monitoring apparatuses to execute:
processing for causing an icon display unit to display a plurality of sensor icons corresponding to respective sensors distances of which from the motion state monitoring apparatus are each within a predetermined distance among the plurality of sensors;
processing for receiving a setting operation on the sensor icons displayed on the icon display unit; and
processing for making, in accordance with the setting operation, the sensor corresponding to the sensor icon correspond to the body part of the subject to which the sensor is to be attached.
